(19) United States
(12) Patent Application Publication
(10) Pub. No.: US 2004/0111109 A1
(43) Pub. Date: Jun. 10, 2004

(54) NASAL VESTIBULUM DILATING PROSTHESIS

(76) Inventor: Jose Francisco Padilla Ruiz, Granada (ES)

Correspondence Address: DALINA LAW GROUP, P.C., 7910 IVANHOE AVE. #325, LA JOLLA, CA, 92037, US

(21) Appl. No.: 10/707,207

(22) Filed: Nov. 26, 2003

(30) Foreign Application Priority Data
Dec. 9, 2002 (ES) U 200202952

(51) Int. Cl. A61M 29/00

(52) U.S. Cl. 606/199

(57) ABSTRACT

Embodiments of the invention relate to a nasal vestibulum dilating prosthesis, for the purpose of configuration as a simple dilating speculum supported by a flange that rests over the nasal dorsum and by another adjustable part which is supported by the head, providing a firm support and allowing the user to benefit from its dilating effects when participating in physical activities, such as sports, as well as during sleep, providing a minimum amount of discomfort and unpleasant effects for the user.

### BACKGROUND OF INVENTION

1. Field of the Invention

This invention has applications in the industry dedicated to the manufacture of apparatus, devices and auxiliary elements for the medical and surgical fields.

2. Description of the Related Art

The Applicant has been familiar for many years with nasal dilating prostheses that, with different designs, obtained the desired effect or at least were able to facilitate the user's breathing; however, they lacked appropriate means of support and would fall rather frequently, thus impeding or making more difficulty any physical activity. Even more important, they could fall during sleep which could cause its inhalation and endanger the life of the user. For these reasons, these devices have been slowly removed from the market, mostly those of small size which lack proper support for both valves.

Actually, the most common solution for this problem consisted of supporting both valves with an elastic lower flange, which is the one that exerts the dilating pressure.

The above mentioned lower elastic flange lacks any means of adjustment and provides an inefficient configuration, as well as discomfort due to excessive pressure. Because of to its size, however, it eliminates those possible inhalation accidents, but it does not have adequate support and it becomes unattached rather easily during the involuntary movements that occur during sleep, as well as during sports and other physical activities.

The problem also affects the dorsal traction dilating adhesive tapes which, in addition to their high cost, lack the necessary efficiency due to the diminished adhesion caused by sweating and, therefore, their use has been sharply reduced.

The best solution to the problems that currently affect these types of devices would be to have an invention that would eliminate all of the above mentioned problems and that, at the same time, would allow a proper and efficient use without the danger of accidents while maintaining is proper positioning at all times.

However, the Applicant is not presently aware of the existence of an invention that presents the above mentioned desirable characteristics.

### SUMMARY OF INVENTION

Embodiments of the invention relate to a nasal vestibulum dilating prosthesis, for the purpose of configuration as a simple dilating speculum supported by a flange that rests over the nasal dorsum and by another adjustable part which is supported by the head, providing a firm support and allowing the user to benefit from its dilating effects when participating in physical activities, such as sports, as well as during sleep, providing a minimum amount of discomfort and unpleasant effects for the user.

The nasal vestibulum dilating prosthesis, proposed by this invention, comprises a small dilating speculum for both valves that leans on the nasal valves and keeps them open through the action of two force vectors, one towards the nasal dorsum and the other, adjustable, surrounding the head by means of an elastic band that is placed over the ears and provides a parallel traction in the same direction as that of the nasal wing elevating muscle, whose action should be reinforced.

In other words, the nasal vestibulum dilating prosthesis, which is the object of this invention, comprises two small handles of approximately 1 cm wide and 1.5 cm high which, leaning over the front two thirds of the internal face of the front nasal valve, produces a dilating action due to the forces created by the means of fixation, presenting two possibilities.

The first means of attachment comprises the support for the frontal branches that hold the dilating plate surrounding the nasal rim and forming a flexible dorsal arc, allowing the accommodation of a wide variety of nose shapes and sizes and having a multiple function.

Using these support means, it is possible to maintain the dilating valve in the most forward position possible, providing better stiffness and volume to the prosthesis and obtaining a nasal support that is similar to that of reading glasses, creating a pair of forces that, in combination with the upper rear traction means, provide an optimum resulting force to obtain the desired dilating pressure.

The second means of attachment is created by attaching the device to the rear surface of the valves, using a stem that is similar to the frontal branch that surrounds the nasal rim, becoming external approximately at the union of the front two thirds with the rear one third and terminating in a ring that holds an elastic band of small cross-section, which is placed around the head above the ears and optimally positions the device, providing an adjustable traction due to the elasticity of the band, which supplies the desired dilating effect and prevents the prosthesis from falling or losing its effectiveness, as well as reducing to a minimum the possibilities of accidents.

### BRIEF DESCRIPTION OF DRAWINGS

To complement this description and to help the understanding of the characteristics of this invention, two figure sheets are attached to this description for illustrative, but not limiting, purposes and which represent the following:FIG. No. 1.—It shows a frontal view of the object of this invention, corresponding to a nasal vestibulum dilating prosthesis.

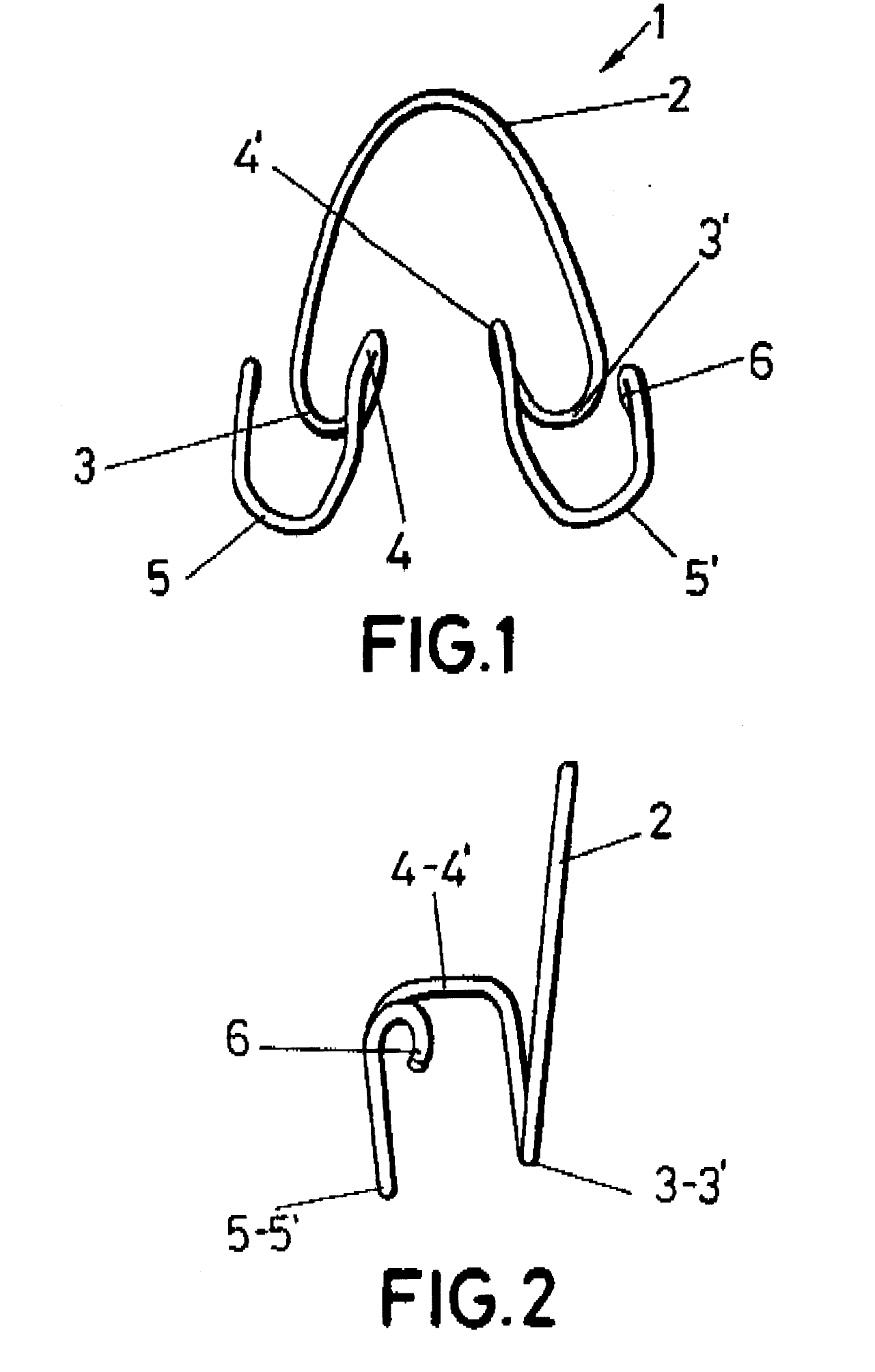

FIG. No. 2.—It shows a lateral elevation of the object previously shown in FIG. 1.

FIG. No. 3.—It shows the invention placed on the nose of the user.

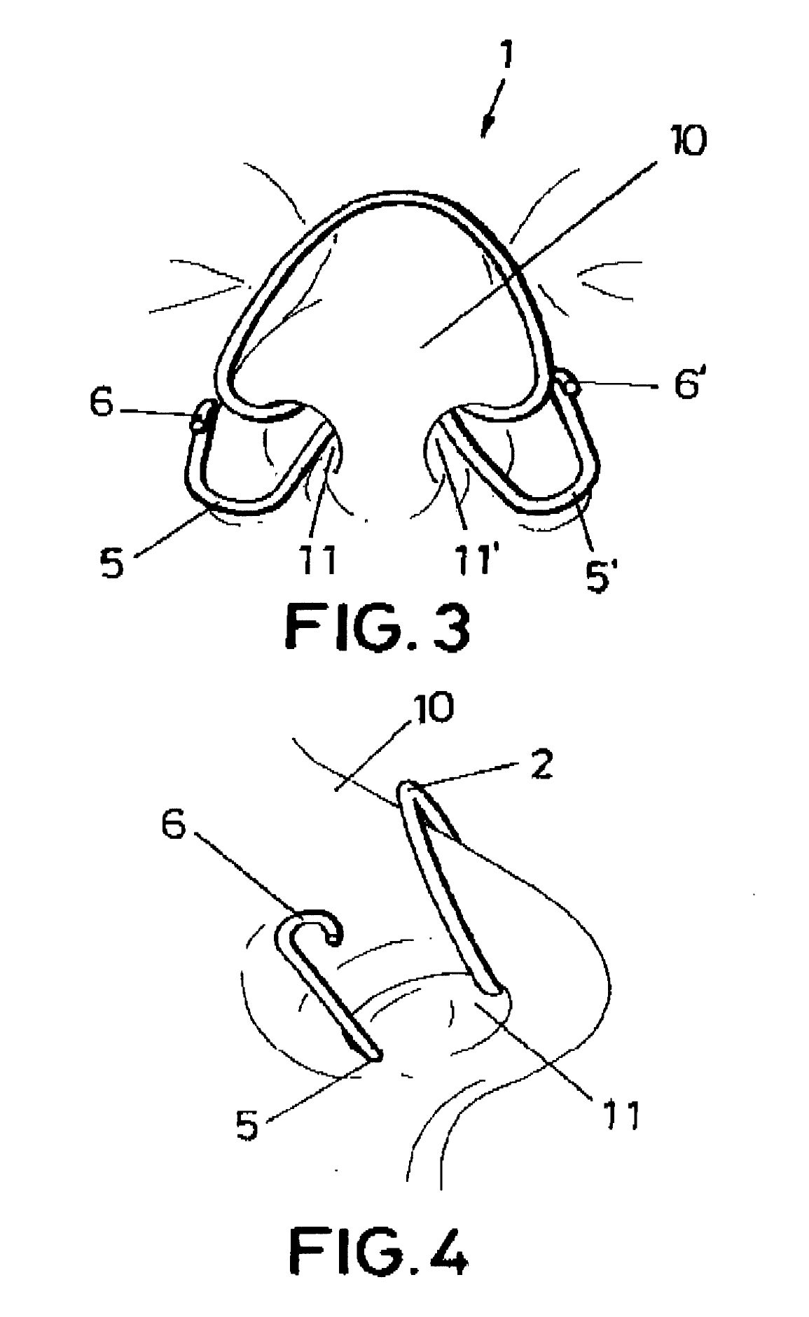

FIG. No. 4.—This illustration shows a side view of the object shown in FIG. 3.

### DETAILED DESCRIPTION

Embodiments of the invention relate to a nasal vestibulum dilating prosthesis, for the purpose of configuration as a simple dilating speculum supported by a flange that rests over the nasal dorsum and by another adjustable part which is supported by the head, providing a firm support and allowing the user to benefit from its dilating effects when participating in physical activities, such as sports, as well as during sleep, providing a minimum amount of discomfort and unpleasant effects for the user.

The nasal vestibulum dilating prosthesis, proposed by this invention, comprises a small dilating speculum for both valves that leans on the nasal valves and keeps them open through the action of two force vectors, one towards the nasal dorsum and the other, adjustable, surrounding the head by means of an elastic band that is placed over the ears and provides a parallel traction in the same direction as that of the nasal wing elevating muscle, whose action should be reinforced.

In other words, the nasal vestibulum dilating prosthesis, which is the object of this invention, comprises two small handles of approximately 1 cm wide and 1.5 cm high which, leaning over the front two thirds of the internal face of the front nasal valve, produces a dilating action due to the forces created by the means of fixation, presenting two possibilities.

The first means of attachment comprises the support for the frontal branches that hold the dilating plate surrounding the nasal rim and forming a flexible dorsal arc, allowing the accommodation of a wide variety of nose shapes and sizes and having a multiple function.

Using these support means, it is possible to maintain the dilating valve in the most forward position possible, providing better stiffness and volume to the prosthesis and obtaining a nasal support that is similar to that of reading glasses, creating a pair of forces that, in combination with the upper rear traction means, provide an optimum resulting force to obtain the desired dilating pressure.

The second means of attachment is created by attaching the device to the rear surface of the valves, using a stem that is similar to the frontal branch that surrounds the nasal rim, becoming external approximately at the union of the front two thirds with the rear one third and terminating in a ring that holds an elastic band of small cross-section, which is placed around the head above the ears and optimally positions the device, providing an adjustable traction due to the elasticity of the band, which supplies the desired dilating effect and prevents the prosthesis from falling or losing its effectiveness, as well as reducing to a minimum the possibilities of accidents.

Manner of fabrication of this invention

It can be seen from the figures that the nasal vestibulum dilating prosthesis (1) comprises a monoblock piece fabricated using a biocompatible rigid and elastic material, preferably of a metallic nature, which may be substituted by an appropriate plastic material with polished surfaces that would prevent any lateral or dorsal decubitus.

When the prosthesis (1) is fabricated using a metallic material, such material shall be an elastic, stainless steel wire, with or without plastic covering. If the invention is fabricated using a plastic material, such material shall be thermo malleable, allowing a perfect adaptation to the user by matching the dorsal support arc, both in angle and curvature.

The fabrication of the prosthesis with a plastic material may be done by using a mold and a single tapping of the molten material, completely avoiding any burrs, irregularities or deformations that could produce undesirable effects. The plastic prosthesis must be transparent or have a color similar to that of the skin.

As can be seen from the illustrations, the prosthesis (1) has a central zone (2) that adopts an arched configuration and which is placed on top of the nasal valve (10), having in the lower ends of the zone (2) two curved configurations (3) and (3′) from where two protuberances (4) and (4′) extend towards the front. From the ends of these protuberances, two other similarly curved protuberances (5) with a concave configuration support two other zones (6) and (6′) over which the suspender will be attached.

The suspender (not shown) could comprise an elastic cord hooked at the ends of (6) and (6′), but this cord may be substituted with any other means of dorsal-cephalic attachment.

1] a nasal vestibulum dilating prosthesis designed to facilitate the entrance of air into the body of a user through a nasal valve (11), comprising a monoblock piece (1) optionally fabricated using a metallic or plastic material produced in only one tapping operation and having a central zone (2), which is situated over the external surface of the nasal pyramid (10) and which has two curved configurations (3) and (3′) at each end, and from where two protuberances (4) and (4′) project towards the internal zone of this device, having concave ends (5) and (5′) and terminating in two semi annular zones (6) and (6′) wherein a support may be attached:

2] The nasal vestibulum dilating prosthesis, in accordance with claim 1 configured with said support, which is attached to annular ends (6) and (6′), further comprising a dorsal-cephalic attachment.

3] The nasal vestibulum dilating prosthesis, in accordance with claim 1, configured with ends (6) and (6′) attached to an adjustable elastic cord, which is placed over the lateral and posterior surfaces of said user's head with a dorsal support band.

4] The nasal vestibulum dilating prosthesis, in accordance with claim 3 configured with said support, which is attached to annular ends (6) and (6′), further comprising a dorsal-cephalic attachment.
